(19) United States
(12) Patent Application Publication
(10) Pub. No.: US 2025/0302365 A1
(43) Pub. Date: Oct. 2, 2025

(54) Methods to Diagnose Small and Large Fiber Neuropathy

(71) Applicant: MORGAN STATE UNIVERSITY, Baltimore, MD (US)

(72) Inventor: Timothy Meeker, Ellicott City, MD (US)

(21) Appl. No.: 19/095,682

(22) Filed: Mar. 31, 2025

Related U.S. Application Data
(60) Provisional application No. 63/571,836, filed on Mar. 29, 2024.

(51) Int. Cl. A61B 5/383; A61B 5/00; A61B 5/372

(52) U.S. Cl. CPC A61B 5/383; A61B 5/372; A61B 5/7267

(57) ABSTRACT

A method for the detection of small fiber and/or large fiber neuropathy comprising application of a near-infrared laser to the area of suspected small fiber or large fiber neuropathy and known healthy areas, simultaneously collecting corresponding brain signal EEG data from the patient, followed by application of electrical stimuli to suspected areas of neuropathy and healthy areas simultaneously collecting corresponding EEG data from the patient. The EEG data for both tests are then run through a neural network trained on a large database of confirmed diagnoses and associated EEG data to make a statistical determination whether the patent exhibits 1) large fiber neuropathy only, 2) small fiber neuropathy only, 3) small and large fiber neuropathy, or, 4) no signs of neuropathy.

### FIELD OF THE INVENTION

This invention relates to methods to diagnose neuropathies, particularly those associated with diabetes.

### BACKGROUND OF THE INVENTION

Loss of sensation and the sense of pain (peripheral neuropathy) occur in about half of patients with adult-onset diabetes. Up to 40 million Americans have had signs of peripheral neuropathy, and 1.4 million additional Americans are diagnosed with diabetes every year. Diabetic peripheral neuropathy can lead to diabetic foot ulcers. Non-healing wounds often lead to loss of toes and feet called non-traumatic amputations. These wounds are often persistent and fail to evoke warning pain responses due to underlying neuropathy. In medical practice, physicians look at diabetic patients' feet periodically to detect these ulcerations and may probe patients' feet with blunt plastic rods to detect peripheral neuropathy. Unfortunately, once patients lose protective sense of touch, their neuropathy is irreversible, and the likelihood of ulceration is high. Amputation may result as a consequence of diabetes and neuropathy.

The neurophysiologic examination of small fibers in peripheral neuropathy (small fiber neuropathies) uses painful infrared laser stimuli to specifically activate Aδ-and C-fibers. However, non-Hispanic black (NHB) subjects have been systematically excluded from all pain studies using painful laser stimuli, due to purported safety concerns.

### SUMMARY OF THE INVENTION

To address this gap in research and diagnosis and treatment of subjects with darkly pigmented skin, this invention presents a validated method for differential diagnosis of small fiber and large fiber peripheral neuropathies. While this invention was initially developed to address the failure of modern science to provide validated diagnosis of peripheral neuropathies in subjects with darkly-pigmented skin, this invention can be used with equal effectiveness for the differential diagnosis of subjects with lightly-pigmented skin.

The present invention describes methods to diagnose neuropathies, especially for Type V and Type VI skin types (on the Fitzpatrick scale) for which little or no data or approach exists. The established approach for other skin types involves a form of painful laser stimuli in individuals that effectively activates Aδ-and C-fibers.

The primary innovation of this method is the application of a near-infrared to the hands or feet or the anatomical area of suspected small fiber or large fiber neuropathy to detect small fiber neuropathy, simultaneously collecting corresponding brain signal EEG data from the patient. Large fiber neuropathy is then detected using von Frey hairs, that produce a mildly painful electrical stimuli, likewise simultaneously collecting corresponding EEG data from the patient. The EEG data for both tests are then run through a neural network trained on a large database of confirmed diagnoses and associated EEG data to make a statistical determination whether a patient exhibits 1) large fiber neuropathy only, 2) small fiber neuropathy only, 3) small and large fiber neuropathy, or, 4) no signs of neuropathy.

### DETAILED DESCRIPTION OF THE INVENTION

As an initial step in validating the present invention, skin temperature responses generated by painful infrared laser stimuli were evaluated in healthy subjects with darkly pigmented skin compared to healthy subjects with lightly pigmented skin to challenge the accepted notion in the prior art that infrared laser stimuli for the diagnosis of small fiber neuropathies could not be used safely with subjects having darkly-pigmented skin.

A 10 mm diameter 1340 nanometer wavelength laser with impulse intensities of 12, 13, 14, and 15 joules of 20 msec duration was used to produce distinctly painful stimuli on the forearms of study subjects. Exactly one half of the study subjects had lightly pigmented skin, while exactly one half of the study subjects had darkly pigmented skin. A thermographic camera recorded skin temperature at 30 Hz after each painful laser impulse. For each laser energy intensity, temperature data was analyzed using a linear mixed model including factors for skin pigmentation, time, and the interaction pigmentation by time.

Contrary to prevailing views, the study results showed the skin of darkly-pigmented subjects had lower response temperatures during a five second post treatment observation period as compared to the skin of lightly-pigmented subjects (temperature light>dark model contrast: Z=3.038; p=0.0024). Importantly, the five darkly pigmented individuals skin temperatures never exceeded 50° C. in these experiments with most stimuli reported as painful. Surprisingly, the study suggests that darkly pigmented skin dissipates heat at a more rapid rate than lightly pigmented skin and that painful laser stimulation using a 1340 nm laser is safe in darkly pigmented participants. Accordingly, the invention presents for the first time a method for diagnosing small fiber neuropathy in patients with darkly-pigmented skin which includes the application of a painful infrared laser to regions of suspected neuropathy.

Large fiber neuropathy is detected according to known methods, typically using von Frey hairs to introduce electrical stimuli to the subject's skin producing a mildly pushing or pressure-like sensation.

The invention further includes the collection of EEG data of the patients during testing for both small fiber and large fiber neuropathies. The EEG data is then run through a trained neural network model trained on data from healthy patients and patients with confirmed types of neuropathies to discover patterns in activating Aδ- and C-fibers that distinguish patients with diabetic peripheral neuropathy from those without neuropathy and to distinguish patients with painful diabatic peripheral neuropathy from those with painless diabetic peripheral neuropathy.

According to a preferred embodiment, EEG data is collected from a patient using 4 to 128 EEG channels while stimulating the anatomical site of suspected neuropathy with a mildly painful or warm laser with an output in the range of 1064 nm to 1500 nm in a patient with any level of pigmentation. Additional EEG data is collected during this laser stimulation of a control anatomical region (e.g. a mirrored region or a region more proximal to the body where patients can distinctly feel a warm or painful sensation from the laser stimulus). A minimum of 10 stimuli are conducted and the EEG potentials from each site is averaged.

The procedure is repeated a minimum of 10 times using mildly painful electrical stimuli in place of the laser stimuli, and the EEG potentials averaged.

The collected EEG potentials for the patient are applied to a statistical model, for example a lasso regression or a machine learning model, developed on patients confirmed to have 1) large fiber neuropathy only, 2) small fiber neuropathy only, 3) small and large fiber neuropathy and, 4) no signs of neuropathy, to make a statistical determination if the patient has abnormalities in the Aβ (large fiber), Aδ (small fiber) or c-fiber (small fiber). In general, marked reduction in the EEG potential in the 20 ms to 150 ms range indicates a potential abnormality in AB fibers, a marked reduction in the EEG potential in the 175 to 600 ms range indicates a potential abnormality in Aδ fibers, and a marked reduction in the EEG potential over the range of 650 to 1400 ms indicates an abnormality in c fibers, see Table 1.

TABLE 1

| | EEG evoked potential | | |
|---|---|---|---|
| Fiber type | temporal range | Abnormality and stimulus type | Suspected Diagnosis |
| A beta (AB) | 20 to 150 ms | Reduction in evoked EEG | Large fiber Neuropathy |
| | | potential in time range in | or large and small fiber |
| | | response to electrical stimuli | neuropathy |
| A delta (A8) | 175 to 600 ms | Reduction in evoked EEG | Small fiber neuropathy |
| | | potential in time range in | or large and small fiber |
| | | response to electrical stimuli | neuropathy |
| | | and laser stimuli | |
| C | 650 to 1400 ms | Reduction in evoked EEG | Small fiber neuropathy |
| | | potential in time range in | or large and small fiber |
| | | response to electrical stimuli and | neuropathy |
| | | laser stimuli | |

The trained neural network includes a classifier that addresses the above time ranges of evoked potential to distinguish patients from each group from each other. Differential diagnosis requires 1) large fiber neuropathy only to have abnormalities in Aβ, but not Aδ or C fiber conduction, 2) small fiber neuropathy only to have abnormalities in Aδ or C fiber, but not Aβ fiber conduction, 3) large and small fiber neuropathy to have abnormalities in both Aβ and Aδ or C fiber conduction and 4) no neuropathy to have no abnormalities in any fiber conduction range.

Notwithstanding the specific embodiments, features, elements, combinations and sub-combinations disclosed herein, it is expressly considered and here disclosed that every single element, every single feature, and every combination and sub-combination thereof disclosed herein may be combined with every other element, feature, combination and sub-combination disclosed herein.

It will be appreciated by those skilled in the art that changes could be made to the embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A method for the detection of small fiber and large fiber neuropathies, comprising:
a. stimulating on a patient a first set of one or more anatomical sites of suspected neuropathy with a laser with an output in the range of 1064 nm to 1500 nm,
b. stimulating on the patient a first set of one or more known healthy sites with the laser with an output in the range of 1064 nm to 1500 nm
c. simultaneously with steps a and b, collecting electroencephalogram data from said patient using 4 to 128 EEG channels,
d. repeating steps a through c a minimum of 10 times,
e. stimulating on the patient a second set of one or more anatomical sites of suspected neuropathy with a painful electrical stimulus,
f. stimulating on the patient a second set of one or more known healthy sites with the painful electrical stimulus,
g. simultaneously with steps e and f, collecting electroencephalogram data from said patient,
h. repeating steps e through g a minimum of 10 times,
i. running accumulated electroencephalogram data collected from repeated steps c and g through a neural network trained on data of diagnosed neuropathy patients and healthy patients and corresponding electroencephalogram data to make a statistical determination whether the patient has small fiber neuropathy, large fiber neuropathy, both small fiber and large fiber neuropathy or no neuropathy.

2. The method according to claim 1, wherein the patient has darkly pigmented skin.

3. The method according to claim 1, wherein, marked reduction in the EEG potential in the 20 ms to 150 ms range indicates a potential abnormality in Aβ fibers,

4. The method according to claim 1, wherein a marked reduction in the EEG potential in the 175 to 600 ms range indicates a potential abnormality in Aδ fibers,

5. The method according to claim 1, wherein a marked reduction in the EEG potential over the range of 650 to 1400 ms indicates an abnormality in c fibers.
